(19) United States
(12) Patent Application Publication
(10) Pub. No.: US 2024/0349999 A1
(43) Pub. Date: Oct. 24, 2024

(54) REMOTELY OPERATED HAND-HELD BRONCHOSCOPE

(71) Applicant: Gregory Brian Bowles, Fenton, MI (US)

(72) Inventor: Gregory Brian Bowles, Fenton, MI (US)

(21) Appl. No.: 18/636,658

(22) Filed: Apr. 16, 2024

Related U.S. Application Data
(60) Provisional application No. 63/460,085, filed on Apr. 18, 2023.

(51) Int. Cl. A61B 1/267; A61B 1/00; A61B 1/005; A61B 1/008

(52) U.S. Cl. CPC A61B 1/2676; A61B 1/00066; A61B 1/00147; A61B 1/0057; A61B 1/008

(57) ABSTRACT

Described herein are embodiments directed to a remotely controlled bronchoscope arrangement that uses an off-the-shelf hand-held bronchoscope to provide distance between a medical professional and a patient. The bronchoscope arrangement comprises an off-the-shelf hand-held bronchoscope that is remotely controlled by cables that mechanically drive the general movement of the bronchoscope. The bronchoscope generally comprises an insertion tube that extends from a handle, which is bent via an insertion tube articulating actuator and rotated by a rotatable insertion tube cogwheel. The rotatable insertion tube cogwheel is configured to rotate the insertion tube about an axis at least where the insertion tube connects to the handle. The rotatable insertion tube cogwheel positions an insertion tube tip angularly about the axis.

### CROSS-REFERENCE TO RELATED APPLICATIONS

This application claims priority to and the benefit of U.S. Provisional Patent Application No. 63/460,085 entitled: MEDICAL ENDOSCOPE HOLDER WITH REMOTE MECHANICAL CONTROL FUNCTIONS, filed on Apr. 18, 2023, the entire disclosure of which is hereby incorporated by reference.

### BACKGROUND OF THE INVENTION

### 1. Field of the Invention

The present invention relates generally to non-electrically driven mechanical endoscopes.

### 2. Description of Related Art

Flexible endoscopes are indispensable tools in modern medicine, revolutionizing diagnostic and therapeutic procedures across various medical specialties. These slender, flexible instruments consist of a long, narrow tube equipped with a miniature camera and lighting system, allowing surgeons to visualize and navigate through the body's internal structures with remarkable precision. Unlike rigid endoscopes, flexible endoscopes can bend and maneuver through intricate pathways, reaching deep into organs such as the gastrointestinal tract, respiratory system, and urinary tract. Their flexibility not only enhances patient comfort during procedures but also enables clinicians to access areas that would otherwise be difficult or impossible to reach.

Robotic assisted endoscopes are driven by electronic control mechanisms to navigate and maneuver within the body during diagnostic and therapeutic procedures. Typically, these mechanisms comprise a combination of controls that electrically respond to manual input. At the forefront of endoscope control is the insertion tube, which is often equipped with articulating joints that allow for flexibility and bending in multiple directions. These articulations are electrically controlled by knobs or levers manipulated by the operator, enabling precise navigation through the body's internal passages. More specifically, endoscopes feature motorized steering systems that enable remote manipulation of the insertion tube by the surgeon, providing dexterity and control during procedures.

It is to innovations related to controlling flexible endoscopes that embodiments of the invention are generally directed.

### SUMMARY OF THE INVENTION

The present invention relates generally to non-electrically driven flexible endoscopes.

In that light, certain embodiments of the present invention envision a bronchoscope arrangement comprising a hand-held bronchoscope, a mounting sled, a mechanical driver that is connected to the hand-held bronchoscope via cables, and. With more specificity, the hand-held bronchoscope comprises a handle, an insertion tube extending from the handle, an insertion tube articulating actuator that is configured to bend the insertion tube, and a rotation knob that is centered on an axis. The rotation knob is configured to rotate the insertion tube about the axis. An insertion tube cogwheel is fixedly attached to the rotation knob in a relationship that is surrounding at least a portion of the rotation knob (i.e., the insertion tube cogwheel is sleeved over at least a portion of the rotation knob). The mounting sled is configured to be driven in a fore/aft direction along a sled frame via a sled screw shaft. The mechanical driver comprises an insertion tube articulating knob that is configured to actuate the insertion tube articulating actuator via an insertion tube actuating cable. The mechanical driver further includes an insertion tube rotating knob is configured to rotate the insertion tube cogwheel via an insertion tube rotating cable. In addition, the mechanical driver includes a sled fore/aft knob that is configured to rotate the sled screw shaft via a sled fore/aft cable. The bronchoscope arrangement is devoid of any motors.

Another embodiment of the present invention contemplates a mounting sled arrangement generally comprising a mounting sled, a sled screw shaft, an articulating actuator screw shaft and a driving cogwheel. The mounting sled slidingly is engaged with a sled frame via a sled nut housing, wherein the sled frame extends between a sled frame front and a sled frame rear. The sled screw shaft is captured at either end of the sled screw shaft by the sled frame (which can be rotatingly engaged with sled screw shaft captured bearing sleeves), wherein the sled nut housing is configured to be driven between the sled frame front and the sled frame rear when the sled screw shaft is rotated via a sled fore/aft cable connected thereto. The articulating actuator screw shaft includes a retaining cuff that extends from the mounting sled. The retaining cuff is configured to traverse along the articulating actuator screw shaft when the articulating actuator screw shaft is rotated via an insertion tube actuating cable connected thereto. The retaining cuff is configured to engage an insertion tube articulating actuator of a bronchoscope. The driving cogwheel extends from a cogwheel extension shaft that is connected to the mounting sled. The driving cogwheel is disposed closer to the sled frame front than the sled frame rear. The driving cogwheel is configured to engage an insertion tube cogwheel of the bronchoscope. The articulating actuator screw shaft is configured to rotate the driving cogwheel via an insertion tube actuating cable connected thereto.

Yet another embodiment of the present invention contemplates a method for operating a hand-held bronchoscope remotely, the method comprising remotely bending an insertion tube, remotely rotating the insertion tube and remotely moving the insertion tube in a fore/aft direction. The step for bending an insertion tube, which extends from the hand-held bronchoscope, is accomplished by actuating an insertion tube articulating actuator on the hand-held bronchoscope by rotating a first cable via a first knob on a mechanical driver. The step for rotating the insertion tube is accomplished by rotating an insertion tube cogwheel on the hand-held bronchoscope by rotating a second cable via a second knob on the mechanical driver. The step for moving the insertion tube in a fore/aft direction is accomplished via a sled connected to the hand-held bronchoscope by rotating a third cable via a third knob on the mechanical driver. These steps are devoid of any assistance from an electric motor.

### BRIEF DESCRIPTION OF THE DRAWINGS

FIG. 1 is a line drawing of a bronchoscope arrangement consistent with embodiments of the present invention;

FIG. 2 is a line drawing of a close-up view of the sled arrangement connected to an off-the-shelf hand-held bronchoscope consistent with embodiments of the present invention;

FIG. 3 is a top view line drawing of the bronchoscope arrangement of FIGS. 1 and 2 consistent with embodiments of the present invention;

FIGS. 4A and 4B illustratively depict line drawings of the front and back sides of a mechanical driver consistent with embodiments of the present invention;

FIGS. 5A and 5B are line drawings that illustratively depicts the sled arrangement without the hand-held bronchoscope consistent with embodiments of the present invention; and

FIG. 6 is a block diagram showing a method to use the bronchoscope arrangement consistent with embodiments of the present invention.

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other similar configurations involving the subject matter directed to the field of the invention. The phrases “in one embodiment”, “according to one embodiment”, and the like, generally mean the particular feature, structure, or characteristic following the phrase, is included in at least one embodiment of the present invention and may be included in more than one embodiment of the present invention. Importantly, such phases do not necessarily refer to the same embodiment. If the specification states a component or feature “may”, “can”, “could”, or “might” be included or have a characteristic, that particular component or feature is not required to be included or have the characteristic. As used herein, the terms “having”, “have”, “including” and “include” are considered open language and are synonymous with the term “comprising”. Furthermore, as used herein, the term “essentially” is meant to stress that a characteristic of something is to be interpreted within acceptable tolerance margins known to those skilled in the art in keeping with typical normal world tolerance, which is analogous with “more or less.” For example, essentially flat, essentially straight, essentially on time, etc. all indicate that these characteristics are not capable of being perfect within the sense of their limits. Accordingly, if there is no specific +/− value assigned to “essentially”, then assume essentially means to be within +/−2.5% of exact. The term “connected to” as used herein is to be interpreted as a first element physically linked or attached to a second element and not as a “means for attaching” as in a “means plus function”. In fact, unless a term expressly uses “means for” followed by the gerund form of a verb, that term shall not be interpreted under 35 U.S.C. § 112(f). In what follows, similar or identical structures may be identified using identical callouts.

With respect to the drawings, it is noted that the figures are not necessarily drawn to scale and are diagrammatic in nature to illustrate features of interest. Descriptive terminology such as, for example, upper/lower, top/bottom, horizontal/vertical, left/right and the like, may be adopted with respect to the various views or conventions provided in the figures as generally understood by an onlooker for purposes of enhancing the reader's understanding and is in no way intended to be limiting. All embodiments described herein are submitted to be operational irrespective of any overall physical orientation unless specifically described otherwise, such as elements that rely on gravity to operate, for example.

Described herein are embodiments directed to a remotely controlled bronchoscope arrangement that uses an off-the-shelf hand-held bronchoscope to provide distance between a medical professional and a patient and to provide additional precision, stability and control of the bronchoscope. The bronchoscope arrangement comprises an off-the-shelf hand-held bronchoscope that is remotely controlled by cables that mechanically drive the general movement of the bronchoscope. The bronchoscope generally comprises an insertion tube that extends from a handle, which is bent via an insertion tube articulating actuator and rotated by a rotatable insertion tube cogwheel. The rotatable insertion tube cogwheel is configured to rotate the insertion tube about an axis at least where the insertion tube connects to the handle. The rotatable insertion tube cogwheel positions an insertion tube tip angularly about the axis.

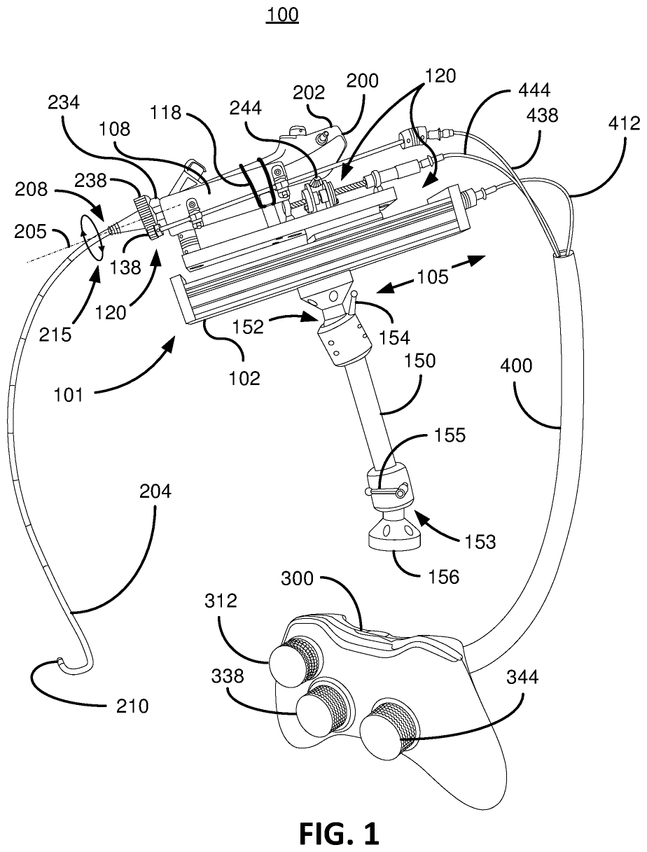

FIG. 1 is a line drawing of a bronchoscope arrangement consistent with embodiments of the present invention. The bronchoscope arrangement 100 can comprise an off-the-shelf (pre-existing) hand-held bronchoscope 200 that is remotely controlled by cables 412, 438 and 444 that mechanically drive the general movement of the bronchoscope 200. The cables 412, 438 and 444 are bundled together in a cable sleeve 400. The hand-held bronchoscope 200 generally comprises an insertion tube 204 extending from a handle 202, wherein the insertion tube 204 is actuated to bend via an insertion tube articulating actuator 244 and rotated by a rotation knob 234 that is retrofitted with a rotatable insertion tube cogwheel 238. The insertion tube cogwheel 238 snuggly and fixedly slides over the rotation knob 234 to provide cog teeth to cooperate with the driving cogwheel 138. The insertion tube articulating actuator 244 causes the insertion tube 204 to bend via a wire (not shown) in the insertion tube 204 when the insertion tube articulating actuator 244 is moved back and forth along the fore/aft direction 105. The rotatable insertion tube cogwheel 238 is engaged with a driving cogwheel 138 and configured to rotate the insertion tube 204 about an axis 205 at least where the insertion tube 204 connects to the handle 202, labeled here as the handle-tube interface 208. The rotatable insertion tube cogwheel 238 positions the insertion tube 204, and more specifically the insertion tube tip 210 angularly about the axis 205, which is shown here spaced away from the axis 205.

In this embodiment, the off-the-shelf bronchoscope 200 is fixedly connected to the mounting sled arrangement 101 via a cradle 108 and retaining strap 118. More specifically, as shown here, the bronchoscope 200 rests inside the cradle 108 and is retained in place (clamped down) by an elastic retaining strap 118. It should be appreciated that there are a variety of other ways to connect an off-the-shelf bronchoscope 200 with the sled arrangement 101, which can include screws, bolts, quick release devices, adhesive, magnetic latches, as well as many other ways known to those skilled in the mechanical arts. The sled arrangement 101 comprises sled motion control elements 120 that control the necessary movement of the insertion tube 204 via the hand-held bronchoscope 200. In this embodiment, the sled motion control elements 120 are controlled by a hand-held mechanical driver 300 that rotates or otherwise twists cables 412, 438 and 444 via knobs 312, 338 and 344 at a hand-held mechanical driver 300. Because the bronchoscope arrangement 100 is driven mechanically by cables 412, 438 and 444 and not by motors, the cost to provide the arrangement 100 is low and power used to control a motor driven bronchoscope is not required satisfying a need for a remote-controlled bronchoscope to be used in low to non-power equipped environments, such as developing countries or ad hoc emergency medical facilities. In this embodiment, the mounting sled arrangement 101 can be attached to a table or platform via an articulating stand 150, such as by a mounting base 156 generically shown here as a ramped foot.

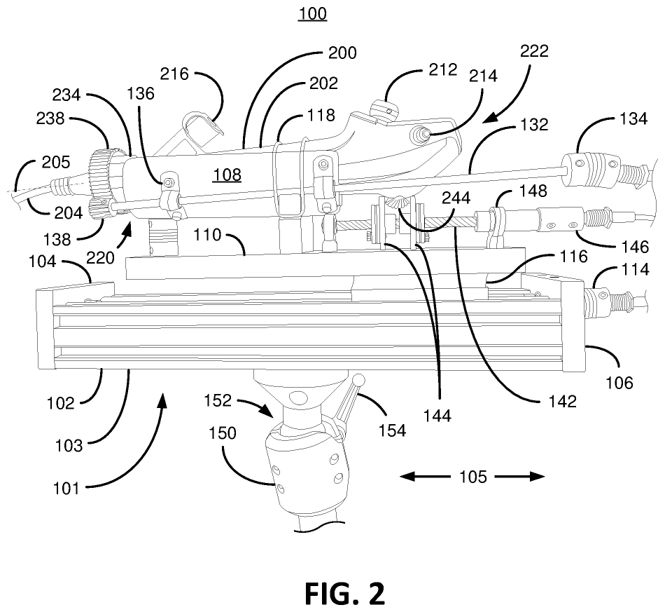

The articulating stand 150 is connected to the sled arrangement 101 to position the sled arrangement 101 in a desired location over a patient. In this embodiment, the articulating stand 150 is mounted to the sled frame bottom 103, as shown in FIG. 2. The articulating stand 150 comprises an upper ball joint 152 at the top that provides freedom of angular rotation to position the sled arrangement 101 over the patient as desired. An upper locking lever arm 154 is levered to lock the sled arrangement 101 in place when rotated in a locking orientation. The stand 150 can be attached to a table, a bed frame, or some other stable surface near the patient. A lower ball joint 153 provides angular freedom to assist in raising and lowering the sled arrangement 101 and can be locked in place via a lower locking lever arm 155 when moved in a desired position.

FIG. 2 is a line drawing of a close-up view of the sled arrangement 101 connected to an off-the-shelf hand-held bronchoscope 200 consistent with embodiments of the present invention. FIG. 2 is described in view of element labeled in FIG. 1. With respect to the depicted off-the-shelf hand-held bronchoscope 200, as shown here it generally comprises an insertion tube 204 that extends from a handle 202 with the ability to steer an insertion tube 204 down a person's bronchial tube. In more detail, the insertion tube 204 is typically used with a fiber optic cable to illuminate past the insertion tube tip 210 in addition to an optical cable to visually show what is in front of the insertion tube tip 210. The bronchoscope 200 has a suction channel 214 (that can connect to a suction tube), a suction valve button 212 and a catheter insertion channel 216, all of which use the tubular path in the insertion tube 204. The insertion tube 204 can be articulated (made to bend) via an internal wire (not shown) by sliding the insertion tube articulating actuator 244 (connected to the internal wire) towards the bronchoscope front 220 and back 222, which in this figure is along the fore/aft direction 105. The insertion tube 204 can be rotated about the axis 205 at the handle-tube interface 208.

As previously discussed, the off-the-shelf hand-held bronchoscope 200 is designed and intended to be held in the hands of a medical professional that uses it directly with a patient, meaning the medical professional is at the bedside of the patient and in close proximity, or otherwise next to the patient wherein the medical professional is able to touch and contact the patient during essentially the entirety of any procedure using the hand-held bronchoscope 200. In contrast, the present embodiment depicts the off-the-shelf hand-held bronchoscope 200 fixedly but removably attached to a sled arrangement 101, which comprises elements in a mechanical drivetrain to remotely control a bronchial procedure via the bronchoscope 200. In this embodiment, the off-the-shelf hand-held bronchoscope 200 is connected to the sled arrangement 101 via a cradle 108 where it is retained in place (clamped down) by a retaining strap 118, which can be an elastic band, that compresses the handle 202 snuggly against the inner channel of the cradle 108. It should be appreciated that there are a variety of other ways to connect an off-the-shelf bronchoscope 200 with the sled arrangement 101, such as screws, bolts, quick release devices, magnets as well as many other ways known to those skilled in the art.

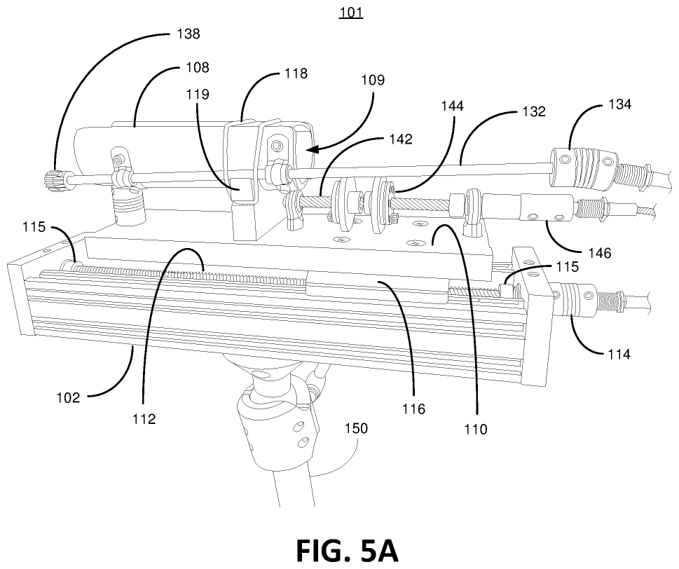

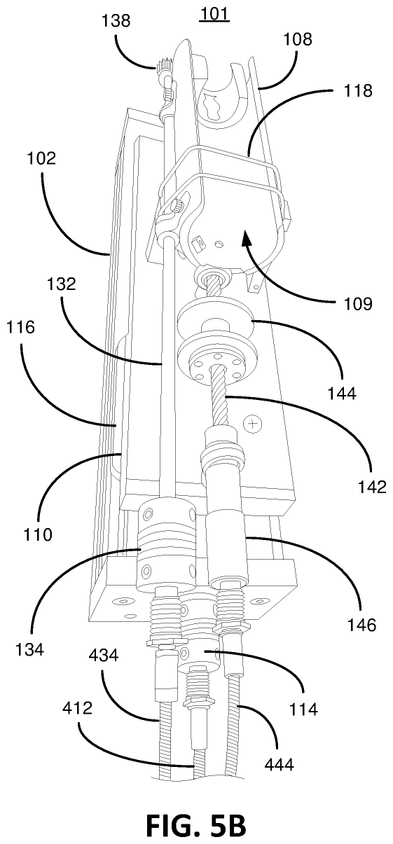

This embodiment depicts the sled arrangement 101 tailored to the depicted off-the-shelf hand-held bronchoscope 200, however other bronchoscope driving/manipulating configurations implemented in an optional sled arrangement can be employed for compatibility with other off-the-shelf hand-held bronchoscopes, endoscopes, and the like, using the similar cable and gear concepts exemplified in this embodiment. Here the sled arrangement 101 comprises a means to remotely move the mounting sled 110 in a fore and aft direction 105 along a sled frame 102. In this embodiment, the mounting sled 110 is connected or otherwise slidingly engaged to the sled frame 102 via a sled nut housing 116 that can be driven to move in the fore and aft direction 105 via a spinning sled screw shaft (which is threaded drive shaft) 112, as shown in FIG. 5A. Certain embodiments contemplate the sled nut housing 116 being a ball nut with a ball return system that uses ball bearings internal to the ball nut to glide the nut housing along the threads of the sled screw shaft when the shaft spins. In operation, the sled fore/aft knob 312 on the mechanical hand-held driver 300 is rotated thereby rotating the sled fore/aft cable 412, which in turn rotates the sled screw shaft 112 via the flexible coupling 114. As the sled screw shaft 112 spins, the sled nut housing 116 is driven in the fore/aft direction 105 defined between the sled frame front 104 and the sled frame rear 106. In this embodiment, a flexible coupling 114 connects the sled screw shaft 112 to the sled fore/aft cable 412. Hence, the off-the-shelf hand-held bronchoscope 200, and more particularly the insertion tube 204, is moved in a fore and aft direction 105 as the mounting sled 110 is moved in the fore and aft direction 105 along a sled frame 102.

The sled arrangement 101 also comprises a means for remotely rotating the insertion tube cogwheel 238 at the bronchoscope front 220 that fixedly fits, or otherwise cuffs over at least a portion of a rotation knob 234 of the bronchoscope 200, as shown. The rotation knob 234 is used to rotate the insertion tube 204 when a medical operator is grasping and rotating the rotation knob 234 when the medical operator is using the hand-held bronchoscope 200 independent of the sled arrangement 101. The insertion tube cogwheel is fitted over the rotation knob 234 to provide cog teeth to interface and cooperate with the driving cogwheel 138 to enable the remotely controlled drivetrain of the driving cogwheel 138 to rotate the rotation knob 234. In this embodiment, the insertion tube cogwheel 238 is driven by a driving cogwheel 138 that meshes with the insertion tube cogwheel 238, which together forms a gear. In operation, the insertion tube rotating knob 338 on the mechanical hand-held driver 300 is rotated thereby rotating the insertion tube rotating cable 438, which in turn rotates the cogwheel extension shaft 132 and therefore the driving cogwheel 138 attached to the distal end of the cogwheel extension shaft 132. Here, the cogwheel extension shaft 132 is supported by the cradle 108 via bearing sleeves 136 that are bolted to the cradle 108. The cogwheel extension shaft 132 is connected to the insertion tube rotating cable 438 via the flexible coupling 134. Hence, the insertion tube 204, is moved clockwise or counterclockwise 215 about the axis 205 as the driving cogwheel 138 drives the insertion tube cogwheel 238.

The sled arrangement 101 further comprises a means for remotely actuating the insertion tube articulating actuator 244 located at the bottom of the handle 108. In this embodiment, the insertion tube articulating actuator 244 is driven by a retaining cuff 144, which comprises a pair of plates that captures or otherwise traps the insertion tube articulating actuator 244. The pair of plates are adjustable to squeeze together and to closely fit around the insertion tube articulating actuator 244. In optional embodiments, the retaining cuff is not a pair of plates but a channeled nut, a cooperating mechanical gripper, or some other retainer that receives the insertion tube articulating actuator 244 in a manner that can retain it and move it as needed. Here, the retaining cuff 144 is threaded along an articulating actuator screw shaft 142 that when spun moves the retaining cuff 144 essentially in the fore and aft direction 105 thereby actuating, or otherwise moving the insertion tube articulating actuator 244. The articulating actuator screw shaft 142 is rotatably retained at either end by bearings 148 where the articulating actuator screw shaft 142 is coupled to the insertion tube actuating cable 444 via a coupling 146. In operation, the insertion tube articulating knob 344 on the mechanical hand-held driver 300 is rotated thereby rotating the insertion tube actuating cable 444, which in turn rotates/spins the articulating actuator screw shaft 142, which drives the retaining cuff 144 back and forth in the fore/aft direction 105. Hence, the insertion tube articulating actuator 244 can be remotely actuated back and forth by driving the retaining cuff 144 along the articulating actuator screw shaft 142 remotely via the insertion tube actuating cable 444.

Certain embodiments of the present invention contemplate a stand 150 connected to the sled arrangement 101 to place the sled arrangement 101 in a desired position over a patient. In this embodiment, only part of the stand 150 that is mounted to the sled frame bottom 103 is shown. As discussed above, the sled frame stand 150 comprises a ball joint 152 that provides freedom of angular rotation to position the sled arrangement 101 over the patient as desired. A locking lever arm 154 is used to lock the sled arrangement 101 in place when levered in a locking orientation. The stand 150 can be attached to a table, a bed frame, or some other stable surface near the patient.

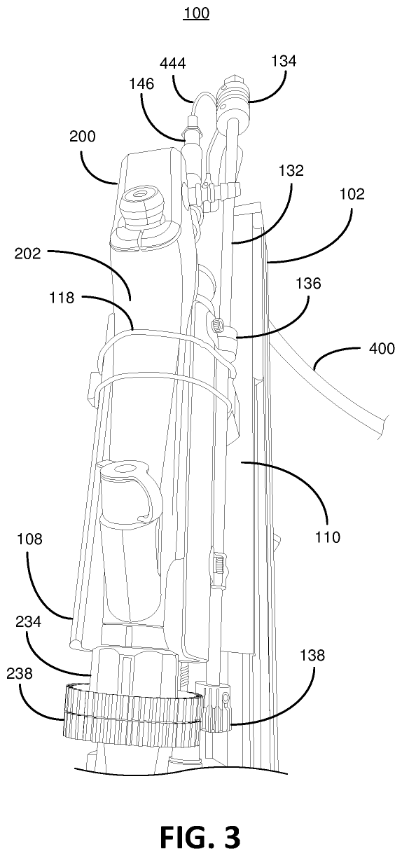

FIG. 3 is a top view line drawing of the bronchoscope arrangement 100 of FIGS. 1 and 2 consistent with embodiments of the present invention. This is another perspective of the hand-held bronchoscope 200 resting inside of the cradle 108 and strapped down therein via the retaining strap 118 extending around the bronchoscope handle 202. Also shown is the cogwheel extension shaft 132, which extends from the flexible coupling 134 and passes through the bearing sleeves 136 and terminates at the driving cogwheel 138, which is meshed in a geared relationship with the insertion tube cogwheel 238. The top of the mounting sled 110 is shown over the sled frame 102. The cable sleeve 400, which bundles and protects the cables 412, 438 and 444, is also labeled along with the insertion tube actuating cable 444 and coupling 146.

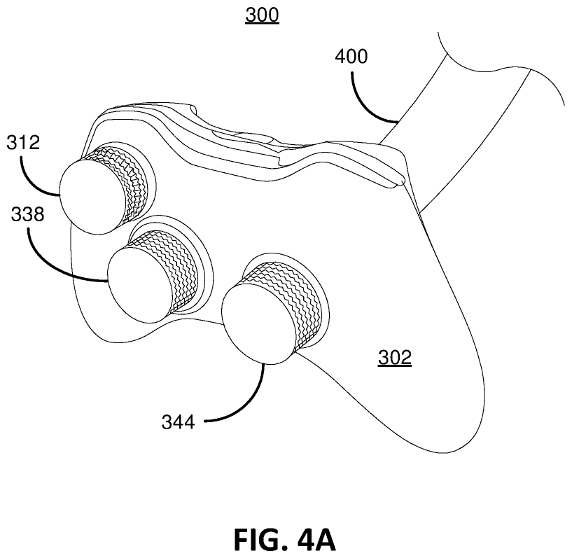

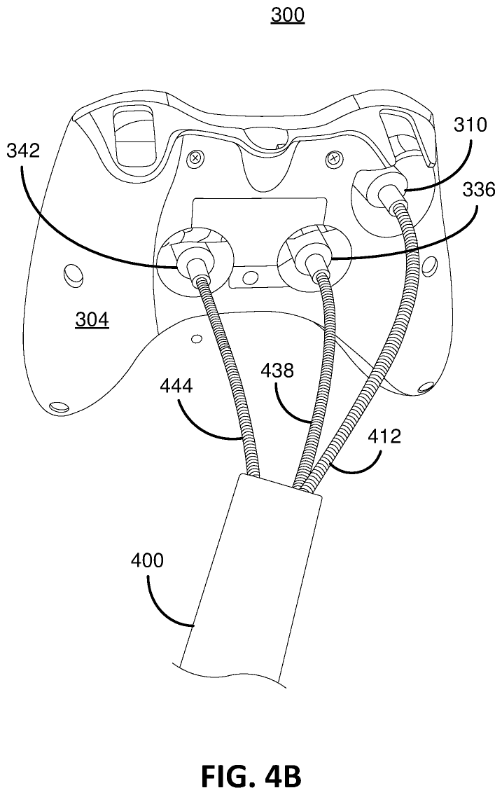

FIGS. 4A and 4B illustratively depict line drawings of the front and back sides of a mechanical driver consistent with embodiments of the present invention. FIG. 4A is a front view of a hand-held mechanical driver 300 that depicts the knobs 312, 338, 344 extending from the front face 302 of the driver 300. Here, the driver 300 has no motors but is rather driven strictly by the power of a human hand turning the knobs 312, 338, 344. The driver 300 is remote from the sled arrangement 101, which for example could be 6 inches away, 3 feet away, 5 feet away, 20 feet away or greater. One advantage of the driver 300 is that the knobs 312, 338, 344 provide tactile feedback to the medical professional when manipulating the hand-held bronchoscope 200 via the sled arrangement 101. More specifically, if the insertion tube tip 210 is encountering an obstruction as it moves down a patient's bronchial tube, such as hitting the patient's tissue, the knob being used will inherently have an increase in resistance signaling to the medical professional that there is a problem. This feedback will immediately and intuitively indicate to the medical professional to back off, reverse, try something different, etc. Hence, feedback through resistance felt by the medical professional in turning a knob 312, 338, 344 can reduce the chance of harm to the patient. Moreover, the remote operation prevents harm to the medical professional should the patient be of risk. Accordingly, the professional can use the bronchoscope arrangement 100 without being in close proximity to the patient, such as in the same room or just several feet, for example 2-3 feet away, from the patient. As shown, the cable sleeve 400 extends from the driver back 304, which houses the mechanical cables 412, 438, 444 connected to the knobs 312, 338, 344. The mechanical driver 300 may include battery operated lights, other indicators, computer chips, a screen for use with a camera, etc.

FIG. 4B illustratively depicts the driver back 304 with the cables 412, 438, 444 extending from the back of the knobs 312, 338, 344. More specifically, the sled fore/aft cable 412 is shown fixedly connected to and extending from the sled fore/aft knob coupler 314, the insertion tube rotating cable 438 is shown fixedly connected to and extending from the insertion tube rotating knob coupler 336, and the insertion tube actuating cable 444 is shown fixedly connected to and extending from the insertion tube articulating knob coupler 342. This is an example of the insertion tube rotating drivetrain. The cables 412, 438, 444 all feed into the cable sleeve 400, as shown. Hence, in operation, when the sled fore/aft knob 312 is spun to the left or right, the sled fore/aft cable 412 spins with it thereby spinning the sled screw shaft 112, which drives the mounting sled 110 fore and aft 105. This is an example of the sled fore/aft drivetrain. Likewise, when the insertion tube rotating knob 338 is spun to the left or right, the insertion tube rotating cable 438 spins with it thereby spinning the cogwheel extension shaft 132, which rotates the driving cogwheel 138. Also likewise, when the insertion tube articulating knob 344 is spun to the left or right, the insertion tube actuating cable 444 spins with it thereby spinning the articulating actuator screw shaft 142, which moves the retaining cuff 144. This is an example of the insertion tube articulating drivetrain.

FIGS. 5A and 5B are line drawings that illustratively depicts the sled arrangement 101 without (devoid of) the hand-held bronchoscope 200 consistent with embodiments of the present invention. From the perspective shown in both FIGS. 5A and 5B, the viewer can see the cradle channel 109 in the cradle 108 configured to receive the hand-held bronchoscope 200. The other elements that cooperate directly with the hand-held bronchoscope 200 are shown, which include the retaining strap 118 hooked to a retaining hook 119 on the side of the cradle 108 (that is used to hold the hand-held bronchoscope 200 in place), the driving cogwheel 138, the cogwheel extension shaft 132 and the flexible coupling 134 as well as the articulating actuator screw shaft 142, the retaining cuff 144 and the coupling 146. FIG. 5A shows the sled screw shaft 112 connected to and in a cooperating relationship with the sled nut housing 116. The sled screw shaft 112 is rotatably retained at the sled frame front 104 and the sled frame rear 106 by respective sled screw shaft capture sleeves 115, which in certain embodiments are bearing sleeves. Both FIGS. 5A and 5B show the mounting sled 110 and the sled frame 102. The sled screw shaft 112 is shown here connected to the flexible coupling 114, which helps accommodate the positioning of the sled frame 102. For clarity, the sled arrangement 101 does not include and is not defined with the articulating stand 150, which is another potential component of the bronchoscope arrangement 100.

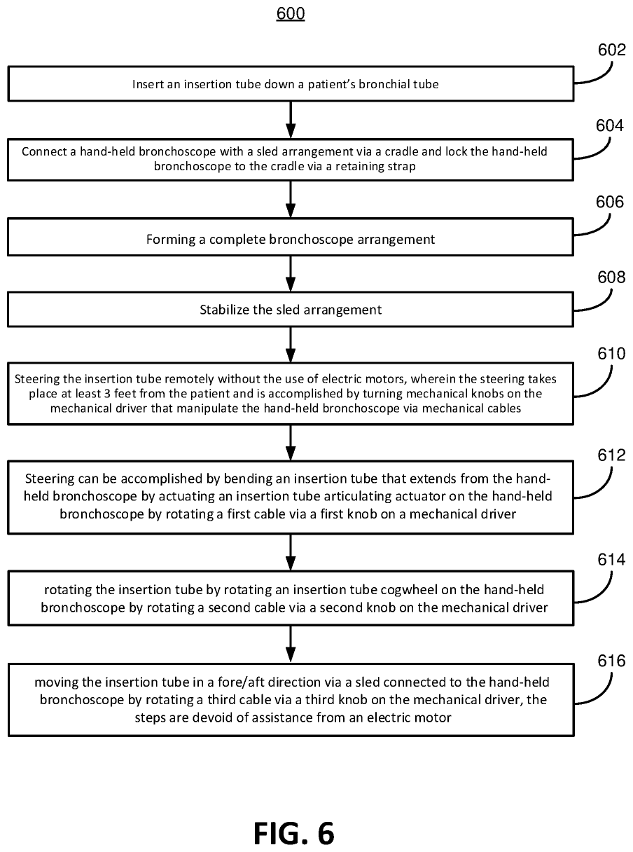

FIG. 6 is a block diagram showing a method to use the bronchoscope arrangement 100 consistent with embodiments of the present invention. FIG. 6 is described in view of at least FIG. 1. As shown in the block diagram 600, certain embodiments of the present invention envision being performed by an operator who first inserts the insertion tube 204 that extends from a hand-held bronchoscope 200 down a patient's bronchial tube, step 602. After the insertion tube 204 is deployed in the patient, the operator connects the hand-held bronchoscope 200 with the sled arrangement 101 via the cradle 108 and locks the hand-held bronchoscope 200 to the cradle 108 via the retaining strap 118 by stretching the retaining strap 118 over the top of the bronchoscope handle 202 and looping the retaining strap 118 over the hook 119 on the side of the cradle 108 (step 204). Connecting the hand-held bronchoscope 200 to the cradle 108 forms the bronchoscope arrangement 100, which combined includes the bronchoscope 200, the sled arrangement 101, and the hand-held mechanical driver 300 that is connected to the sled arrangement 101 via cables 412, 438 and 444 (step 606). An articulating stand 150 can be attached to the sled arrangement 101 to stabilize the sled arrangement 101 when the articulating stand 150 is connected to a table, bed, wall or on the ground, by attaches or otherwise connects, for example, step 608. It should be appreciated that other means for stabilizing the sled arrangement 101 known to those skilled in the art could be used in place of the articulating stand 150. The operator can then steer the insertion tube 204 remotely without the use of electric motors, wherein in this embodiment the steering takes place at least 3 feet from the patient and is accomplished by turning mechanical knobs 312, 338 and 344 on the mechanical driver 300 that manipulate the hand-held bronchoscope 200 via mechanical cables 412, 438 and 444, step 610. With more specificity to the bronchoscope arrangement 100, the operator can bend the insertion tube 204 by actuating an insertion tube articulating actuator 244 on the hand-held bronchoscope 200 by rotating a first cable 444 via a first knob 344 on a mechanical driver 300 (step 612), rotate the insertion tube 204 by rotating an insertion tube cogwheel 238 on the hand-held bronchoscope 200 by rotating a second cable 438 via a second knob 338 on the mechanical driver 300 (step 614), and move the insertion tube 204 in a fore/aft direction 105 via a sled 110 connected to the hand-held bronchoscope 200 by rotating a third cable 412 via a third knob 312 on the mechanical driver 300 (step 616).

It should be appreciated that the exemplified method that is directed to the hand-held bronchoscope 200 of FIG. 1 can conceptionally be accomplished with a different hand-held bronchoscope (or other flexible endoscopes) having various means for articulating and rotating the different hand-held bronchoscope's (or endoscope's) insertion tube. In this scenario, an optional sled arrangement can be provided comprising appropriate elements to operate the various means for articulating and rotating the different hand-held bronchoscope's (endoscope's) insertion tube. Accordingly, though the placement and structure of the appropriate elements used to actuate the various means for articulating and rotating an insertion tube on the sled arrangement may be different, it is envisioned that they will still be driven by rotating at least the cables 412, 438 and 444 using at least the knobs 312, 338 and 344 on the mechanical driver 300 within the scope and spirit of the present invention.

With the present description in mind, below are some examples of certain embodiments illustratively complementing some of the apparatus embodiments discussed above and presented in the figures to aid the reader. Accordingly, the elements called out below are provided by example to aid in the understanding of the present invention and should not be considered limiting. The reader will appreciate that the below elements and configurations can be interchangeable within the scope and spirit of the present invention. The illustrative embodiments can include elements from the figures.

In that light, certain embodiments of the present invention envision a bronchoscope arrangement 100 comprising a hand-held bronchoscope 200, a mounting sled 110, a mechanical driver 300 that is connected to the hand-held bronchoscope 200 via cables 412, 438 and 444. With more specificity, the hand-held bronchoscope 200 comprises a handle 202, an insertion tube 204 extending from the handle 202, an insertion tube articulating actuator 244 that is configured to bend the insertion tube 204, and a rotation knob 234 that is centered on an axis 205. The rotation knob 234 is configured to rotate the insertion tube 204 about the axis 205. An insertion tube cogwheel 238 is fixedly attached to the rotation knob 234 in a relationship that is surrounding at least a portion of the rotation knob 234 (i.e., the insertion tube cogwheel 238 is sleeved over at least a portion of the rotation knob 234). The mounting sled 110 is configured to be driven in a fore/aft direction 105 along a sled frame 102 via a sled screw shaft 112. The mechanical driver 300 comprises an insertion tube articulating knob 344 is configured to actuate the insertion tube articulating actuator 244 via an insertion tube actuating cable 444. The mechanical driver 300 further includes an insertion tube rotating knob 338 is configured to rotate the insertion tube cogwheel 238 via an insertion tube rotating cable 438. In addition, the mechanical driver 300 includes a sled fore/aft knob 312 that is configured to rotate the sled screw shaft 112 via a sled fore/aft cable 412. The bronchoscope arrangement 100 is devoid of any motors.

In an embodiment of the bronchoscope arrangement 100, the handle 202 is envisioned to be mounted to the mounting sled 110 via a cradle 108.

The bronchoscope arrangement 100 further envisions the insertion tube actuating cable 444 being connected to an articulating actuator screw shaft 142, which is engaged with a retaining cuff 144, the retaining cuff 144 engaged with the insertion tube articulating actuator 244, the insertion tube articulating knob 344 is configured to linearly move the retaining cuff 144 along the articulating actuator screw shaft 142 when the insertion tube articulating knob 344 is rotated.

The insertion tube cogwheel 238 of the bronchoscope arrangement 100 is further envisioned to mesh with a driving cogwheel 138 that is configured to rotate by turning the insertion tube insertion tube rotating cable 438 via the rotating knob 338.

The bronchoscope arrangement 100 further contemplates the hand-held bronchoscope 200 being a pre-existing (off-the-shelf) hand-held bronchoscope that is produced and sold as a retail device in the marketplace.

The bronchoscope arrangement 100 further envisions the knobs 312, 338 and 344 being configured to be rotated or otherwise manipulated in some other fashion by a human hand without motors or some other electronic assistance, wherein the knobs 312, 338 and 344 are configured to provide resistance feedback to an operator when the knobs 312, 338 and 344 are rotated by the human hand of the operator.

The mechanical driver 300 of the bronchoscope arrangement 100 can be handheld.

The bronchoscope arrangement 100 can further comprise a stand 150 that mounts the sled frame 102 to a table, a wall, or a bed. Certain embodiments of the stand 150 envision the stand comprising at least one joint 152 configured to positionally adjust the sled frame 102.

Another embodiment of the present invention contemplates a mounting sled arrangement 101 generally comprising a mounting sled 110, a sled screw shaft 112, an articulating actuator screw shaft 142 and a driving cogwheel 138. The mounting sled 110 slidingly is engaged with a sled frame 102 via a sled nut housing 116, wherein the sled frame 102 extends between a sled frame front 104 and a sled frame rear 106. The sled screw shaft 112 is captured at either end 114 of the sled screw shaft 112 by the sled frame 102 (which can be rotatingly engaged with sled screw shaft captured bearing sleeves 115), wherein the sled nut housing 116 is configured to be driven between the sled frame front 104 and the sled frame rear 106 when the sled screw shaft 112 is rotated via a sled fore/aft cable 412 connected thereto. The articulating actuator screw shaft 142 includes a retaining cuff 144 that extends from the mounting sled 110. The retaining cuff 144 is configured to traverse along the articulating actuator screw shaft 142 when the articulating actuator screw shaft 142 is rotated via an insertion tube actuating cable 444 connected thereto. The retaining cuff 144 is configured to engage an insertion tube articulating actuator 244 of a bronchoscope 200. The driving cogwheel 138 extends from a cogwheel extension shaft 132 that is connected to the mounting sled 110. The driving cogwheel 138 is disposed closer to the sled frame front 104 than the sled frame rear 106. The driving cogwheel 138 is configured to engage an insertion tube cogwheel 238 of the bronchoscope 200. The cogwheel extension shaft 142 is configured to rotate the driving cogwheel 138 via an insertion tube actuating cable 444 connected thereto.

The mounting sled arrangement 101 further envisions the cables 412, 438 and 444 being connected to a mechanical driver 300 that is devoid of any motors.

The mounting sled arrangement 101 envisions an embodiment wherein the mechanical driver 300 comprises an insertion tube articulating knob 344 that is configured to actuate the insertion tube articulating actuator 244 via the insertion tube actuating cable 444, an insertion tube rotating knob 338 that is configured to rotate the insertion tube cogwheel 238 via the insertion tube rotating cable 438, and a sled fore/aft knob 312 that is configured to rotate the sled screw shaft 112 via the sled fore/aft cable 412. The embodiment further envisions the knobs 312, 338 and 344 being configured to be rotated by a human hand, such that the knobs 312, 338 and 344 provide resistance feedback to an operator when the knobs 312, 338 and 344 are rotated by the human hand of the operator.

The mounting sled arrangement 101 further envisions the mounting sled 110 comprising the bronchoscope 200 attached thereto, wherein the bronchoscope 200 comprises a handle 202 and an insertion tube 204 extending from the handle 202. The bronchoscope insertion tube articulating actuator 244 is configured to bend the insertion tube 204. The insertion tube cogwheel 238 is centered on an axis. The insertion tube cogwheel 238 is fixedly attached to a rotation knob 234 of the bronchoscope 200, wherein the insertion tube cogwheel 238 is configured to rotate the insertion tube 204 about the axis 205.

Yet another embodiment of the present invention contemplates a method for operating a hand-held bronchoscope 200 remotely, the method comprising remotely bending an insertion tube 204, remotely rotating the insertion tube 204 and remotely moving the insertion tube 204 in a fore/aft direction 105. The step for bending an insertion tube 204, which extends from the hand-held bronchoscope 200, is accomplished by actuating an insertion tube articulating actuator 244 on the hand-held bronchoscope 200 by rotating a first cable 444 via a first knob 344 on a mechanical driver 300. The step for rotating the insertion tube 204 is accomplished by rotating an insertion tube cogwheel 238 on the hand-held bronchoscope 200 by rotating a second cable 438 via a second knob 338 on the mechanical driver 300. The step for moving the insertion tube 204 in a fore/aft direction 105 is accomplished via a sled 110 connected to the hand-held bronchoscope 200 by rotating a third cable 412 via a third knob 312 on the mechanical driver 300. These steps are devoid of any assistance from an electric motor.

The method further envisions that an operator receives resistance feedback when turning the first, the second or the third knob 312, 338 and 344, which may be an indicator that the insertion tube tip 210 of the insertion tube 204 is hitting an obstruction, such as attempting to press through otherwise unharmed tissue along a patient's bronchial tube, for example.

The method further envisions that the cables separate an operator from the hand-held bronchoscope 200 by at least 6 inches but preferably more than 3 feet. It should be appreciated that knobs can be placed anywhere along the chain of rotation (drivetrain) to assist or test out the functionality of the drivetrain. More specifically, rotating any portion of the specific drivetrain will rotate the entire specific drivetrain because it is all connected. Hence, for example with respect to rotating the insertion tube 204, it can be equally done by rotating an insertion tube cogwheel 238 on the hand-held bronchoscope 200, or by rotating the second cable 438 by hand or via a knob attached thereto, or by the second knob 338 on the mechanical driver 300, or by a cogwheel extension shaft 132, or by any portion along the drivetrain of the insertion tube cogwheel 238 because they are all interconnected and will rotate together.

These exemplified embodiments are not exhaustive of the embodiments presented throughout the description, but rather are merely select examples of a contemplated chain of embodiments consistent with embodiments of the present invention. In other words, there are numerous other embodiments described herein that are not necessarily presented in the embodiment examples presented above that would be appreciated by a person understanding the concepts disclosed in this description.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended embodiments are expressed. For example, the orientation of the elements, such as the elements in the insertion tube articulating chain, the elements in the insertion tube rotating chain, the elements in the sled fore/aft chain, etc., can include other geometries not explicitly shown in the embodiments above while maintaining essentially the same functionality without departing from the scope and spirit of the present invention. Likewise, the materials and construction of the mounting sled, cables and mechanical driver can be of various material types without departing from the purpose the scope and spirit of the present invention. It should further be appreciated that the cogs and gears can be replaced with other suitable mechanical motion transfer elements having different shapes and/or constructions while maintaining the described rotational chains to drive a remotely drive a hand-held bronchoscope while staying in the bounds of the primary ideas presented without departing from the scope and spirit of the present invention. It should be further understood that other types of off-the-shelf scopes can be used with the in a similar manner with various constructions of the mounting sled arrangement as disclosed above without departing from the scope and spirit of the present invention. Moreover, though motors are not used to drive the drivetrains in the mounting sled arrangement, lights, batteries, and other electronic elements may be incorporated in the bronchoscope arrangement without departing from the scope and spirit of the present invention.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed.

What is claimed is:

1. A bronchoscope arrangement comprising:
a hand-held bronchoscope comprising:
a handle;
an insertion tube extending from the handle;
an insertion tube articulating actuator configured to bend the insertion tube; and
a rotation knob centered on an axis, the rotation knob configured to rotate the insertion tube about the axis,
an insertion tube cogwheel fixedly attached to the rotation knob and surrounding at least a portion of the rotation knob;
a mounting sled configured to be driven in a fore/aft direction along a sled frame via a sled screw shaft;
a mechanical driver comprising:
an insertion tube articulating knob configured to actuate the insertion tube articulating actuator via an insertion tube actuating cable;
an insertion tube rotating knob configured to rotate the insertion tube cogwheel via an insertion tube rotating cable;
a sled fore/aft knob configured to rotate the sled screw shaft via a sled fore/aft cable;
the bronchoscope arrangement devoid of any motors.

2. The bronchoscope arrangement of claim 1, wherein the handle is mounted to the mounting sled via a cradle.

3. The bronchoscope arrangement of claim 1, wherein the insertion tube actuating cable is connected to an articulating actuator screw shaft, which is engaged with a retaining cuff, the retaining cuff engaged with the insertion tube articulating actuator, the insertion tube articulating knob is configured to linearly move the retaining cuff along the articulating actuator screw shaft when the insertion tube articulating knob is rotated.

4. The bronchoscope arrangement of claim 1, wherein the insertion tube cogwheel meshes with a driving cogwheel configured to rotate by turning the insertion tube insertion tube rotating cable via the rotating knob.

5. The bronchoscope arrangement of claim 1, wherein the hand-held bronchoscope is a pre-existing hand-held bronchoscope.

6. The bronchoscope arrangement of claim 1, wherein the knobs are configured to be rotated by a human hand of the operator.

7. The bronchoscope arrangement of claim 6, wherein the knobs are configured to provide resistance feedback to an operator when the knobs are rotated by the human hand.

8. The bronchoscope arrangement of claim 1, wherein the mechanical driver is handheld.

9. The bronchoscope arrangement of claim 1 further comprising a stand that mounts the sled frame to a table, a wall, or a bed.

10. The bronchoscope arrangement of claim 9, wherein the stand comprises at least one joint configured to positionally adjust the sled frame.

11. A mounting sled arrangement comprising:
a mounting sled slidingly engaged with a sled frame via a sled nut housing, the sled frame extending between a sled frame front and a sled frame rear;
a sled screw shaft captured at either end of the sled screw shaft by the sled frame, the sled nut housing configured to be driven between the sled frame front and the sled frame rear when the sled screw shaft is rotated via a sled fore/aft cable connected thereto;
an articulating actuator screw shaft with a retaining cuff extending from the mounting sled, the retaining cuff is configured to traverse along the articulating actuator screw shaft when the articulating actuator screw shaft is rotated via an insertion tube actuating cable connected thereto, the retaining cuff configured to engage an insertion tube articulating actuator of a bronchoscope; and
a driving cogwheel extending from a cogwheel extension shaft that is connected to the mounting sled, the driving cogwheel disposed closer to the sled frame front than the sled frame rear, the driving cogwheel configured to engage an insertion tube cogwheel of the bronchoscope, the cogwheel extension shaft configured to rotate the driving cogwheel via an insertion tube actuating cable connected thereto.

12. The mounting sled arrangement of claim 11, wherein the cables connected to a mechanical driver that is devoid of any motors.

13. The mounting sled arrangement of claim 11, wherein the mechanical driver comprises:
an insertion tube articulating knob configured to actuate the insertion tube articulating actuator via the insertion tube actuating cable;
an insertion tube rotating knob configured to rotate the insertion tube cogwheel via the insertion tube rotating cable; and
a sled fore/aft knob configured to rotate the sled screw shaft via the sled fore/aft cable.

14. The mounting sled arrangement of claim 13, wherein the knobs are configured to be rotated by a human hand.

15. The mounting sled arrangement of claim 14, wherein the knobs are configured to provide resistance feedback to an operator when the knobs are rotated by the human hand of the operator.

16. The mounting sled arrangement of claim 11, wherein the mounting sled comprises the bronchoscope attached thereto.

17. The mounting sled arrangement of claim 16, wherein the bronchoscope comprises:
a handle;
an insertion tube extending from the handle;
the bronchoscope insertion tube articulating actuator configured to bend the insertion tube; and
the insertion tube cogwheel centered on an axis, the insertion tube cogwheel fixedly attached to a rotation knob of the bronchoscope, the insertion tube cogwheel configured to rotate the insertion tube about the axis.

18. A method for operating a hand-held bronchoscope remotely, the method comprising:
bending an insertion tube that extends from the hand-held bronchoscope by actuating an insertion tube articulating actuator on the hand-held bronchoscope by rotating a first cable via a first knob on a mechanical driver;
rotating the insertion tube by rotating an insertion tube cogwheel on the hand-held bronchoscope by rotating a second cable via a second knob on the mechanical driver; and
moving the insertion tube in a fore/aft direction via a sled connected to the hand-held bronchoscope by rotating a third cable via a third knob on the mechanical driver,
the steps are devoid of assistance from an electric motor.

19. The method of claim 18, wherein the cables separate an operator from the hand-held bronchoscope by at least 3 feet.

20. The method of claim 18, wherein an operator receives resistance feedback when turning the first, the second or the third knob.
